(19) United States
(12) Patent Application Publication
(10) Pub. No.: US 2025/0345127 A1
(43) Pub. Date: Nov. 13, 2025

(54) METHODS AND SYSTEMS FOR TRACKING MULTIPLE OPTICAL MARKERS IN A ROBOTIC SURGICAL PROCEDURE

(71) Applicant: LEM Surgical AG, Bern (CH)

(72) Inventors: Yossi BAR, Muri bei Bern (CH); Matvey RZHAVSKIY, Muri bei Bern (CH); Marcial Patric EGLI, Bern (CH)

(73) Assignee: LEM Surgical AG, Bem (CH)

(21) Appl. No.: 19/277,818

(22) Filed: Jul. 23, 2025

Related U.S. Application Data
(63) Continuation-in-part of application No. PCT/EP2024/052338, filed on Jan. 31, 2024.
(60) Provisional application No. 63/442,457, filed on Jan. 31, 2023.
(60) Provisional application No. 63/781,254, filed on Mar. 31, 2025.

(51) Int. Cl. A61B 34/20; A61B 34/30

(52) U.S. Cl. CPC A61B 34/20; A61B 34/30; A61B 2034/2057; A61B 2034/2065; A61B 2034/2068

(57) ABSTRACT

A robotic surgical system includes a first robotic arm, a second robotic arm, a first camera on the first robotic arm, and a second camera on the second robotic arm. A controller receives images from the first and second cameras, kinematically positions the first and second robotic arms in a surgical coordinate space, kinematically tracks a position of the first camera in the surgical coordinate space and optically tracks a position of the second camera using the first camera. Positions of secondary markers in a field of view of the second camera are calculated based on the position of the second camera as tracked by the first camera.

### CROSS-REFERENCE

This application is a continuation-in-part of PCT Application No. PCT/EP2024/052338, filed Jan. 31, 2024, which claims the benefit of U.S. Provisional Application No. 63/442,457, filed Jan. 31, 2023; this application also claims the benefit of U.S. Provisional No. 63/781,254, filed Mar. 31, 2025; the entire content of each of which are incorporated herein by reference.

### BACKGROUND

### 1. Field of the Invention

The disclosed technology relates to systems and methods for surgical robotic registration and navigation. More particularly, the disclosed technology relates to systems and methods which use navigation markers attachable to bony and other patient anatomies.

Surgical and other robotic systems often utilize a camera or sensor to track objects in a robotic space surrounding the surgical robot. In some surgical robotic procedures, radiopaque (RO) markers may be attached to a patient's bony or other anatomy, and the patient may be imaged by computerized tomography (CT) scanning, and the marker locations used to “register” the image of the patient in the robotic surgical space. For example, as taught in commonly owned PCT application PCT/IB2022/052297 (published as WO2022/195460), the full disclosure of which is incorporated herein by reference, one or more RO markers which are shown in the patient CT scan may be screened by a camera which is located on an arm of a multi-armed surgical robot to establish an initial position of the marker in the robot's surgical space which has a coordinate system defined relative to the robot's chassis or cart. During the subsequent surgical procedure, changes in the patient's position can be calculated based upon observed changes in the marker positions over time.

The surgical robots described in PCT publication WO2022/195460 and other surgical robots commonly in use typically rely on a single camera and a single marker to track patient position during robotic surgery. While workable, the use of a single marker and single camera requires a relatively large marker as the camera will not always be sufficiently close to, or in proper alignment with, the marker to allow markers with a reduced size. Said another way, the marker target needs to be large to allow the camera to accurately determine its location from a distance.

In presently available robotic surgical systems, navigation markers typically measure at least 7 cm to 15 cm across order to provide the necessary 1 mm to 2 mm accuracy at the tool tip when the camera is placed at a standard 1.5 m to 2.5 m from the patient and patient marker. Such large markers are disadvantageous as they can interfere with surgeon view of and access to the surgical site and they can easily deflect under their own weight, causing a loss of accuracy.

The use of registration markers as a first step in a robotic surgical procedure is well known. This step is carried out in order to register the coordinate system of a robotic surgical system, such as a spinal surgical robotic system, with the patient's anatomy. Navigation markers may then be positioned in place of the registration marker to synchronize the robotic navigation system with the patient's anatomy.

While generally successful, the use of both registration and navigation markers does carry some drawbacks. Firstly, the duplication adds time, cost and complexity to the procedure. Second, performing a CT scan for registration exposes the patient to radiation.

Thus, it would be desirable to provide improved robotic surgical systems and methods. In particular, it would be desirable to provide robotic surgical systems and methods that do not require an initial registration step for robotic navigation or for any other purpose. It would be further desirable to provide surgical robotic systems and methods which allow for viewing and tracking of relatively small markers which do not interfere with the surgical procedure without any substantial loss of accuracy or precision. It would be still further desirable that the surgical robotic systems and methods allowed for placement of multiple navigation markers even after the start of a surgical procedure. At least some of these objectives will be met by the inventions described and claimed herein.

### SUMMARY

The systems and methods of the disclosed system address the shortcomings addressed above. By employing multiple, repositionable navigation cameras and/or other sensors and multiple navigation markers affixed at different locations on the patient, smaller navigation markers can be used while maintaining accurate scanning and/or tracking of the patient anatomy. In some embodiments, multiple robotic arms may be operated in a surgical field with at least one arm holding a navigation camera or other sensor and at least one other arm holding a tool or end effector, where the arms are manipulated by a controller of the robotic system. Such systems and methods are useful in orthopedic procedures where the markers are affixed to the patient's bony anatomy, for example individual vertebra of the patient's spine.

While the disclosed technology will find particular use with optical cameras, the principles of the technology can be applied to any sensing technology, being particularly useful to sensing technologies that limited to line-of-sight visibility and/or by proximity between sensor and marker. Suitable sensing technologies include laser scanning or tracking, such as light detection and ranging sensors (LIDAR), magnetic sensing, scanning and tracking; ultrasound sensing, scanning, and tracking, and the like.

In some embodiments, placement of multiple “miniature” markers on a patient's bony anatomy will allow a small navigation camera (also referred to herein as a secondary camera) mounted on a surgical robot arm, often together with a tool or an end effector, to access and track regions of a surgical field which would be inaccessible to a larger navigation camera (also referred to herein as a primary camera) mounted on a dedicated surveillance arm. Accordingly, systems and methods are presented for affixing multiple miniature markers, sometimes referred to herein as secondary markers, on a patient's bony anatomy, where the miniature markers can be tracked by one or more small navigation cameras mounted on robotic arms which carry tools and or end effector that are used to perform the procedure.

In some embodiments, the small or secondary camera(s) may be detachably mounted on the surgical robotic arms (e.g., they may be add-on devices) while the larger or primary camera may be attached to a dedicated surveillance arm. The larger or primary camera can be configured to track a larger or primary navigation marker attached, providing a surveillance view of most or all of surgical field. In addition, the primary camera can track the secondary camera(s) so that the position of the secondary markers in the surgical field can be tracked by kinematically tracking a position of the primary camera (based upon the kinematics of the surveillance arm) and optically tracking the position(s) of the secondary camera(s) using the primary camera. All movements, tracking, and calculations may be performed by the robotic controller.

In surgical robotic systems according to the disclosed technology, the multiple surgical robotic arms may be mounted on a single chassis (typically a single mobile chassis or cart). The phrase a “single chassis” means that the chassis, when present under the operating table, provides a single, rigid platform which in turn provides a single surgical coordinate space. For example, the “single chassis” may comprise two, three, or more mobile or other components, subassemblies, or the like, which may be joined in situ beneath the table to form a single chassis in accordance with the disclosed technology. In other examples, such separate components, subassemblies, or the like, may be pre-assembled at the surgical site or elsewhere before being moved to a location beneath the surgical table. While such single chassis will usually have a unitary construction, in other instances, the platforms my comprise two, three, or more component structures which are assembled in situ at the surgical site.

The multiple surgical robotic arms may carry and deploy various surgical tools, end effectors, navigation cameras, and the like, and a robotic controller. The system may include a display and user interface mounted on or in the single chassis. The controller may automatically control the movement of some or all of the surgical robotic arms, surveillance arm(s), and other robotic system components, based upon the information provided by the primary and secondary navigation cameras. In some embodiments, the controller may display images from the cameras, allowing the surgeon to manually control some or all of the surgical tools or end effectors.

The robotic systems of the disclosed technology are advantageous as the multiple navigation cameras do not interfere with surgeon line of sight and workflow and can be optimally placed for patient safety. For example, a larger primary navigation camera can be positioned away from the surgical site where the procedure is being performed while the multiple secondary or “miniature” navigation markers can be placed inside the human body where interference the procedure is minimized. While the primary camera will often not be able to view the secondary navigation markers, the secondary camera can view and track the secondary markers while the secondary camera itself can be tracked by the primary camera.

This approach may be useful with robotic spinal surgery where individual vertebra often misalign during a procedure, such as pedicle screw placement on multiple vertebras for fusion or other purposes. By placing secondary markers on at least some of the vertebra, the misalignments can be tracked, and the robotic arms repositioned during the procedure.

Accordingly, provided herein are systems and methods for accurate surgical navigation in a robotic surgical system, optionally a robotic system for spinal surgery. In some embodiments, the accurate navigation system is provided in the context of a multi-arm surgical robotic system comprising at least two robotic arms. In one such multi-arm surgical robotic system, at least one arm is responsible for surgical tasks and at least one arm is used to carry and operate at least one camera as part of a robotic navigation system. The at least two robotic arms are optimally mounted on a single chassis that houses a central controller that governs movement of the robotic arms. In an alternate embodiment, the multi-arm surgical robotic system mounted on a single chassis may have at least three arms, wherein at least two arms are responsible for surgical tasks and at least one arm is used to carry and operate at least one camera as part of a robotic navigation system. One of skill in the art will understand that, for present purposes of disclosing systems and methods for accurate surgical navigation, it is also possible to conceive of a surgical robotic system wherein multiple surgical arms are mounted on a single chassis and wherein a navigation arm is brought into the surgical field on a separate cart or chassis, with communication and coordination being provided between the surgical arm chassis and the navigation chassis. The skilled artisan will understand the advantages and disadvantages of this configuration as compared to robotic systems where all arms, including the navigation arm, are based on a single chassis with a central controller. Multiple robotic arms (including navigation arms) could be brought to the surgical field on individual carts in an alternative configuration for use with the disclosed technology, but that this may have disadvantages when compared with a single chassis design.

In some embodiments, one arm of the surgical robotic system may hold a conventional or “primary” navigation camera on a “surveillance arm” and one or more additional arms of the surgical robotic system may hold a tool or end effector. These arms are often referred to as a “working arm.” “operating arm.” or “tool arm.” In accordance with the disclosed technology, a secondary, typically smaller, camera may be attached to one or of the other robotic arms, often together with tools or end effectors which are also held the robotic arms. The other arms will be deployed closer to the surgical site as a matter of course during the surgical procedure and will thus be able to get close enough to track the secondary markers with minimal additional interference with the procedure.

In some embodiments, the smaller navigation camera may be held by a “dedicated” robotic arm which is not holding a surgical tool. This arrangement may be desirable when an additional arm is available as it allows optimal positioning of the secondary navigation camera as it may be positioned in the surgical field without regard to the placement or operation of the surgical tool.

In some embodiments, the secondary or “miniature” navigation markers that may be placed directly on portions of a patient's anatomy, for example on the vertebrae of a patient during a robotic spinal surgery procedure. The markers may optionally incorporate radiopaque elements that would render them suitable for use in a conventional initial registration step in a robotic surgical procedure, but this is not necessary in an embodiment where a separate conventional registration step has already been carried out. The miniature markers may also be deployed in a system that incorporates one or more separate registration markers. The miniature markers may be visible to the conventional navigation camera on the surveillance arm or to the smaller navigation camera on the end effector arm, or to both cameras.

In some embodiments, small navigation markers may also be placed directly on a tool or end effector mounted on a robotic arm functioning primarily to carry out surgical steps in a robotic surgical procedure. In particular embodiments, small navigation markers may be placed on the secondary navigation cameras that are themselves attached to the surgical arms, often to the tools or end effectors on the surgical arms of the surgical robotic system. In this way, the secondary cameras can be optically racked by the primary camera. In some embodiments, the secondary camera could be kinematically tracked based on the position of the supporting robotic arm, but kinematic tracking can be less accurate and more difficult to implement.

In some embodiments, the secondary navigation cameras are attached to surgical arms, usually to the tools/end effectors, and are visible to a primary navigation camera held by a dedicated surveillance or other robotic arm at a convenient distance from the surgical field (usually 0.5 m to 1.5 m). This allows for an integrated approach wherein the end-effector-mounted secondary navigation camera can be positioned to have an optimal view of miniature secondary navigation markers placed on the patient anatomy. The primary navigation camera maintains an overall view of the surgical field which, importantly, includes the secondary navigation camera(s).

The robotic controller is configured to kinematically coordinate movement of all of the robotic arms (both the surveillance arm and the working arms) with respect to each other and the patient's anatomy without necessarily requiring initial registration of the coordinate system of the miniature markers with the anatomy of the patient. One of skill in the art will understand that this coordination of robotic arms with system navigation is also possible in an embodiment where there is one surveillance arm, one operating arm (without a miniature navigation camera being mounted on it) and one additional robotic arm holding a small navigation camera close to the surgical field, so long as the this small navigation camera has a suitable miniature marker attached to it.

As a further advantage, the methods of the disclosed technology do not require conventional registration of the secondary markers, although conventional registration of the secondary markers could be performed in certain circumstances. Registration of the primary marker with the patient's pre-op computed tomography (CT) or other scan, in contrast, will usually still be performed. Elimination of the need to register the secondary markers is advantageous, for example, because the secondary markers will often be placed during the procedure so would not be in place for a pre-op scan. For example, in procedures on a patient's spine, the secondary markers will often be placed only after the procedure has begun and the surgical site progressively opened.

The disclosed technology, however, allows the secondary cameras to “optically register” the secondary markers as they are implanted during a procedure. After each secondary marker is attached to an exposed bony structure or other anatomy, the primary camera scans the secondary marker, and the controller can “register” the optically determined marker position in the surgical coordinate space. As the primary marker will usually have been conventionally registered with the pre-op image, the controller can then relate the secondary marker positions to the image. More importantly, the secondary cameras will be able to track the secondary markers during the course of the procedure to determine how their relative positions may change as, for example, individual vertebra torque relative to each other and change alignment.

According to an embodiment of the disclosed technology, a larger, conventional navigation marker may be placed on anatomy of interest of a patient, for example on bony anatomy of the patient and, in a particular example, on a vertebra of the spine of the patient. This marker may have radiopaque elements and may thus be used in a conventional registration step using a CT scan. This initial registration step serves to register the navigation component of the robotic system to the anatomy of the patient, in a representative example to an aspect of the patient's bony anatomy, or specifically a vertebra of the patient's spine. Upon registration, the navigation system is then registered to the bony anatomy of the patient and can track that anatomy using, for example, the conventional navigation camera held by the surveillance arm of the robotic system described herein in an embodiment of the disclosed technology.

In similar embodiments, secondary navigation markers may be placed on the patient's anatomy of interest without pre-registration and often after the procedure has been commenced. The anatomy of interest could be bone, skin, soft tissue or, in the specific example provided, adjacent areas of the patient's spine. These secondary navigation markers are not connected to the primary (usually larger) registration marker and will often be outside of the of the primary navigation camera's field of view. The secondary markers may be registered to the anatomy of interest using a secondary navigation camera that is typically held by a working robotic arm. The secondary navigation camera can be positioned to view the secondary navigation marker. The primary navigation camera held on the surveillance arm views both the primary navigation marker (used in the initial, conventional registration) and the navigation marker on the secondary camera (or arm holding the secondary camera), while the secondary camera views the surgical field and the secondary navigation markers which will often not be visible to the primary navigation camera on the surveillance arm. Thus, the central controller of the robotic system can register the secondary markers to the anatomy of interest through this navigation “loop” (chain of navigation registrations).

In a first aspect of the disclosed technology, a method for performing robotic surgical procedure comprises providing a surgical robot with at least a first robotic arm, a second robotic arm, a first camera on the first robotic arm, a second camera on the second robotic arm, and a controller configured to receive images from the first and second cameras and to kinematically position the first and second robotic arms. A primary marker is placed at a primary location on an anatomy of a patient, and the patient anatomy and the primary marker are scanned with the first camera to generate a primary image. The controller registers the location of the primary marker within a coordinate system of the surgical robot based upon the primary image, and one or more secondary markers are placed on secondary location(s) on the patient anatomy. The one or more secondary markers are scanned with the second camera to generate a secondary image, and the controller registers the secondary locations relative to the primary location within the coordinate system of the surgical robot based upon the secondary image.

In some instances, registering the secondary locations relative to the primary location comprises determining the location of the secondary camera relative to the primary camera.

In some instances, determining the location of the secondary camera relative to the primary camera comprises scanning the secondary camera with the primary camera.

In some instances, determining the location of the secondary camera relative to the primary camera comprises the controller kinematically determining the locations of the first and second robotic arms.

In some instances, the methods herein further comprise continuing to scan the primary marker with the first camera during subsequent portions of the robotic surgical procedure to track the primary location in the coordinate system of the surgical robot.

In some instances, the methods herein further comprise continuing to scan the secondary marker(s) with the second camera during subsequent portions of the robotic surgical procedure to track the secondary location(s) in the coordinate system of the surgical robot.

In some instances, tracking the secondary location(s) in the coordinate system of the surgical robot comprises tracking the position of the second camera relative to the first camera.

In some instances, the primary marker is larger than the secondary markers and the primary camera when scanning the primary marker is at a distance from the primary marker which is greater than a distance of the secondary camera from the secondary marker(s) when scanning the secondary marker(s).

In some instances, at least first and robotic arms are mounted on a common chassis which establishes the coordinate system.

In some instances, the primary marker is affixed to a primary vertebra and the secondary markers are affixed to secondary vertebra.

In some instances, the surgical tools are operated by one or more of the robotics arms.

In a second aspect of the disclosed technology, a method for performing a robotic surgical procedure comprises providing a surgical robot with at least a first robotic arm, a second robotic arm, a first camera on the first robotic arm, a second camera on the second robotic arm, and a controller configured to receive images from the first and second cameras and to kinematically position the first and second robotic arms in a surgical coordinate space. A location of the first camera is kinematically tracked in the surgical coordinate space, and a location of the second camera is optically tracked in the surgical coordinate space with the first camera. Location(s) in the surgical coordinate space of one or more secondary markers affixed at secondary location(s) on the patient anatomy ate optically tracked with the second camera, and the controller calculates the location(s) in the surgical coordinate space of the one or more secondary markers based the kinematically tracked position of the first camera and the optically tracked locations of the secondary markers relative to the second camera.

In some instances, the secondary markers are in a field of view of the secondary camera but not in a field of view of the first camera.

In some instances, the methods of the disclosed technology further comprise tracking a location in the surgical coordinate space of a primary marker affixed at a primary location on an anatomy of a patient with the first camera located a first distance from the primary marker.

In some instances, a first distance between the first camera and the primary marker is larger than a second distances between the second camara and the secondary markers and the primary marker is larger than the secondary markers.

In some instances, the first camera is located at a distance of up to 1.5 m from the patient anatomy and the second camera is positioned at a distance of 30 cm or less from the target anatomy.

In some instances, the primary marker has an area an area greater than 10 cm2 and the secondary markers have an area less than 10 cm2.

In some instances, the methods of the disclosed technology further comprise continuing to scan the primary marker with the first camera during subsequent portions of the robotic surgical procedure to track the primary location in the coordinate system of the surgical robot.

In some instances, the methods of the disclosed technology further comprise continuing to scan the secondary marker(s) with the second camera during subsequent portions of the robotic surgical procedure to track the secondary location(s) in the coordinate system of the surgical robot.

In some instances, the at least first and robotic arms are mounted on a common chassis which establishes the coordinate system.

In some instances, the primary marker is affixed to a primary vertebra and the secondary markers are affixed to secondary vertebra.

In some instances, the methods of the disclosed technology further comprise performing a procedure on at least one of the primary and secondary vertebra using surgical tools operated by one or more of the robotics arms.

In a third aspect of the disclosed technology, a robotic surgical system comprises at least a first robotic arm, a second robotic arm, a first camera on the first robotic arm, and a second camera on the second robotic arm; and a controller configured to (a) receive images from the first and second cameras, (b) kinematically position the first and second robotic arms in a surgical coordinate space. (c) kinematically track a position of the first camera in the surgical coordinate space. (d) optically track a position of the second camera using the first camera, and (e) calculate positions of secondary markers in a field of view of the second camera based on the position of the second camera being tracked by the first camera.

In some instances, the robotic surgical systems further comprise a surgical tool deployed on the second robotic arm.

In some instances, the robotic surgical systems further comprise an optical marker attached proximate the second camera configured to allow optical tracking of the second camera by the first camera.

In some instances, the first camera is configured to be positioned at a distance of at least 1.5 m from the patient anatomy and the second camera is configured to be positioned at a distance of 30 cm or less from the patient anatomy.

In some instances, the surgical robot comprises at least a third robotic arm which carries a surgical tool and the first robotic arm comprises a surveillance robot arm which carries only the first camera.

In some instances, the robotic surgical systems further comprise the third robotic arm also carries a third camera with a marker that allows optical tracking by the first camera.

In some instances, the first camera is configured to track a primary marker affixed to the patient anatomy.

In some instances, at least the first and second robotic arms are mounted on a single chassis which defines the surgical coordinate space.

In some instances, the robotic surgical systems further comprise all robotic arms and the controller are mounted on the single chassis.

In some embodiments, control of the surgical robots of the disclosed technology may also rely on “pose” information provided through a registration step for planning and/or control. The disclosed technology provides for improved placement and design of one or more navigation cameras, as discussed herein, and the methods and systems disclosed technology can be applied to a wide variety of different surgical robot architectures and designs.

One of skill in the art will understand that specific examples have been given with reference to adjacent portions of the bony anatomy (spine) of a patient. However, the present system and method can be used with respect to adjacent portions of any anatomy of interest (bone, skin, soft tissue) because the small navigation markers can be placed randomly without a separate registration requirement. Instead, they are registered to the anatomy of interest through the disclosed navigation loop.

In some embodiments, another type of navigation loop can be described. Similarly, to the previous one only this time there is no marker on the miniature navigation camera. This time this camera is positioned in a known predefined location on one of the surgical arms and the central controller can use its robotic known location in space to be calculated in the overall navigation/registration loop.

In a fourth aspect of the disclosed technology, a surgical marker comprises a base having an attachment interface and a bone anchor. The bone anchor is configured to be removably attached to or implanted in a patient's bony anatomy, and a trackable marker structure is configured to be attached to and repeatably removed and reattached to the attachment interface in a fixed orientation.

As described in more detail below, the ability to remove and replace the trackable marker portion of the surgical marker allows a user to improve the line-of-sight viewing during a robotic surgical operation when that marker is not needed for navigation or other purposes while relacing the trackable marker when needed for navigation or other purposes. To allow such removal and replacement, it is necessary that the base and the trackable marker elements have an attachment interface that preserves a precise orientation between the base and the trackable marker even after successive removals and replacements.

While these surgical markers will find particular use as secondary markers in the systems and methods described previously, the surgical markers may also find use as primary markers or in any other situations where trackable markers, fiducials, or the like, may be used in robotic surgery.

The bone anchor base will typically have a very low profile so that the trackable marker may be removed when the selective marker might interfere with imaging or other access to a surgical field. An interface between the bone anchor base and the trackable marker is configured to allow detachment and reattachment of the trackable marker to the bone anchor base with a precisely repeatable orientation. That is, the orientation of the bone anchor base and the trackable marker will remain constant even after successive detachments and reattachments.

While the initial orientation of the bone anchor relative trackable marker (before implantation of the surgical marker) will often be precisely fixed and known to the surgical robotic system, knowledge of the initial orientation is not necessary as the location of the trackable marker in a robotic coordinate system can be determined by scanning of a surgical field after implantation of the surgical marker. It is necessary only that the orientation of the trackable marker after reattachment be precisely the same as the initial orientation at the time of initial implantation.

In some instances, the bone anchor of the bone anchor base may comprise a post configured to be directly or indirectly implanted in bone. For example, the bone anchor comprises a pin having a self-penetrating end configured to be penetrated into the bony anatomy.

In other instances, the bone anchor may comprise a clamp configured to be fixedly located on the bony anatomy without penetration.

In some instances, the trackable marker structure comprises one or more trackable features on a plate. For example, the trackable marker structure may comprise a planar array of one or more radiopaque features or of one or more optically visible features.

In some instances, the bone anchor may have an attachment axis and the planar array may be removably attached to the bone anchor at an angle in a range from 20° to 90° relative to the attachment axis, often 30° to 60° relative to the attachment axis.

In some instances, the bone anchor may be configured to upwardly protrude from a surface of the patient's bony anatomy by a distance no greater than 5 cm after attachment of the bone anchor to the bony anatomy.

In some instances, the attachment interface may include at least one connecting element configured to detachably mate with one or more corresponding connecting features on the trackable marker structure. For example, the at least one connecting element and the at least one corresponding connecting element may comprise magnetic or magnetizable element(s) to provide detachable attachment.

In such instances, the at least one connecting element and the at least one corresponding connecting element further comprise mechanical locators to provide repeatable alignment.

In some instances, the at least one connecting element and the at least one corresponding connecting element may comprise mechanical elements to provide both detachable attachment and repeatable alignment.

In some instances, the base may be free from radiopaque and optical trackable features as it will usually be unnecessary to scan the base itself.

In a fifth aspect of the disclosed technology, methods for performing a surgical procedure on a patient using a surgical robot having (a) one or more robotic arms controlled in a robotic coordinate system by a controller and (b) at least one sensor for observing a surgical field comprise implanting primary and surgical markers. A primary surgical marker may be implanted at a primary location on the patient's bony anatomy, and a surgical marker may be implanted at a secondary location on the patient's bony anatomy. The primary and surgical markers are typically registered in the robotic coordinate system, and a trackable marker structure may be detached from the surgical marker to leave a bone anchor in place at the secondary location on the patient's bony anatomy. A first surgical procedure is then performed with the controller controlling movement of the at least one robotic arm, and the sensor observes the surgical field while the trackable marker structure of the surgical marker is removed, and the bone anchor remains in place. In this way the secondary marker's observable presence in the surgical field is minimized or eliminated, i.e., the trackable marker portion has been removed leaving only the low-profile bone anchor in place.

The terms “procedure” and “surgical procedure” as used herein are meant to broadly encompass any specific step or series of steps being performed by a surgical robot as part a robotic surgical operation. For example, a robotic surgical operation may comprise implantation of a spacer between vertebrae while individual procedures performed as part of the operation may include making initial incisions, distraction, spacer placement, closing the incisions, etc. The trackable markers may be detached and reattached at various points during or between such procedures.

In some instances, the methods may further comprise scanning the surgical field with at least one sensor to determine the locations of the primary and surgical markers in the surgical field before detaching the trackable marker structure. In such instances, the methods may still further comprise reattaching the trackable marker structure to the bone anchor of the surgical marker and rescanning the surgical field with the at least one sensor to track the locations of the primary and surgical markers after performing the first surgical procedure to determine changes in the patient anatomy.

In some instances, the methods may yet further comprise detaching the trackable marker structure from the surgical marker to leave the bone anchor in place after rescanning the surgical field with the at least one sensor and performing a second surgical procedure. Such methods may still further comprise reattaching the trackable marker structure to the bone anchor of the surgical marker and rescanning the surgical field to track the locations of the primary and surgical markers after performing the second surgical procedure to determine changes in the patient anatomy.

These previously described steps may be repeated to perform additional surgical procedures.

In some instances, the surgical markers may comprise a base which includes the bone anchor and an attachment interface. For example, the attachment interface may include at least one connecting element configured to detachably mate with one or more corresponding connecting features on the trackable marker structure, and the at least one connecting element and the at least one corresponding connecting element may comprise magnetic or magnetizable elements to provide detachable attachment, where the at least one connecting element and the at least one corresponding connecting element may further comprise mechanical locators to provide repeatable alignment. The at least one connecting element and the at least one corresponding connecting element may further comprise mechanical elements to provide both detachable attachment and repeatable alignment.

In some instances, the trackable marker of the secondary marker may be at least partly mechanically attached to the bone anchor.

In other instances, the trackable marker of the secondary marker may be at least partly magnetically attached to the bone anchor. For example, the trackable marker of the secondary marker may be magnetically attached to and mechanically aligned with the bone anchor

In some instances, methods may further comprise scanning the patient with a registration camera or other sensor to produce a registration image while the surgical marker has the trackable marker attached.

In some instances, after implantation of the surgical marker, the bone anchor may protrude upwardly from a surface of the patient's bony anatomy by a distance no greater than 5 cm, often being 3 cm or less.

### INCORPORATION BY REFERENCE

### Description of the Background Art

WO2022/195460 has been described above. Other commonly owned publications and applications include PCT/IB2022/052297 (published as WO2022/195460); PCT/2022/058988 (published as WO2023/067415); PCT/IB2022/058972 (published as WO2023/118984); PCT/IB2022/058982 (published as WO2023/118985); PCT/IB2022/058978 (published as WO2023/144602); PCT/IB2022/058980 (published as WO2023/152561); PCT/IB2023/055047 (published as WO2023/223215); PCT/IB2022/058988 (published as WO2023/237922); PCT/IB2023/055439; PCT/IB2023/056911; PCT/IB2023/055662; PCT/IB2023/055663; U.S.63/524,911; and U.S.63/532,753, the full disclosures of which are incorporated herein by reference.

The full disclosures of commonly owned PCT Applications PCT/EP2024/052373, entitled “SINGLE ORIGIN MARKER ASSEMBLIES AND METHODS FOR THEIR USE.” and PCT Application PCT/EP2024/052353, entitled “INTEGRATED MULTI-ARM MOBILE MODULAR SURGICAL ROBOTIC SYSTEM.” are incorporated herein by reference in their entirety.

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosed technology are set forth with particularity in the appended claims. A better understanding of the features and advantages of the disclosed technology will be obtained by reference to the following detailed description that sets forth illustrative embodiments, in which the principles of the disclosed technology are utilized, and the accompanying drawings of which:

FIG. 1 shows a surgical robotic system comprising a primary navigation camera mounted on a robotic surveillance arm and a secondary navigation camera mounted on a robotic working arm that also carries a surgical tool, in accordance with some embodiments.

FIG. 2 shows a surgical robotic system comprising a primary navigation camera mounted on a primary robotic surveillance arm and a secondary navigation camera mounted on a dedicated secondary robotic arm that carries only the secondary camera, in accordance with some embodiments.

FIG. 3 is a top plan view of an end effector comprising a secondary navigation camera attached to a gripper tool having an attachment flange which can be removably attached to a remote end of a surgical robot arm, in accordance with some embodiments.

FIGS. 4A and 4B are isometric side views of an end effector having an attachment flange which can be removably attached to a remote end of a surgical robot arm. The end effector carries a secondary navigation camera but is free from any surgical tool. FIG. 4B is rotated 90° about its longitudinal axis relative to FIG. 4A, in accordance with some embodiments.

FIGS. 5A and 5B illustrate a surgical marker in accordance with the principles of the disclosed technologies having an implantable base and a detachable marker shown with the removable marker attached (FIG. 5A) and detached (FIG. 5B).

FIG. 6 illustrates a pattern of connector elements on a top surface of the implantable base of the surgical marker of FIGS. 5A and 5B.

FIG. 6A is a cross-sectional view taken along line 6A-6A of FIG. 6.

FIG. 7 illustrates a pattern of connector elements on a bottom surface of the trackable marker of the surgical marker of FIGS. 5A and 5B.

FIG. 7A is a cross-sectional view taken along line 7A-7A of FIG. 7.

FIGS. 8 and 9 show the effect of line-of sight imaging provided by removal of the trackable marker from the base during a surgery.

### DETAILED DESCRIPTION

Unless otherwise defined, all technical terms used herein have the same meaning as commonly understood by one of ordinary skill in the art to which this disclosure belongs.

As used herein, the singular forms “a.” “an,” and “the” include plural references unless the context clearly dictates otherwise. Any reference to “or” herein is intended to encompass “and/or” unless otherwise stated.

As used herein, the term “about” in some cases refers to an amount that is approximately the stated amount.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The disclosed systems and methods are now described with specific reference to the attached figures. One of skill in the art will realize that the described embodiments are representative in nature and reasonable departures from the described embodiments are still possible while staying within the scope of the disclosed technology.

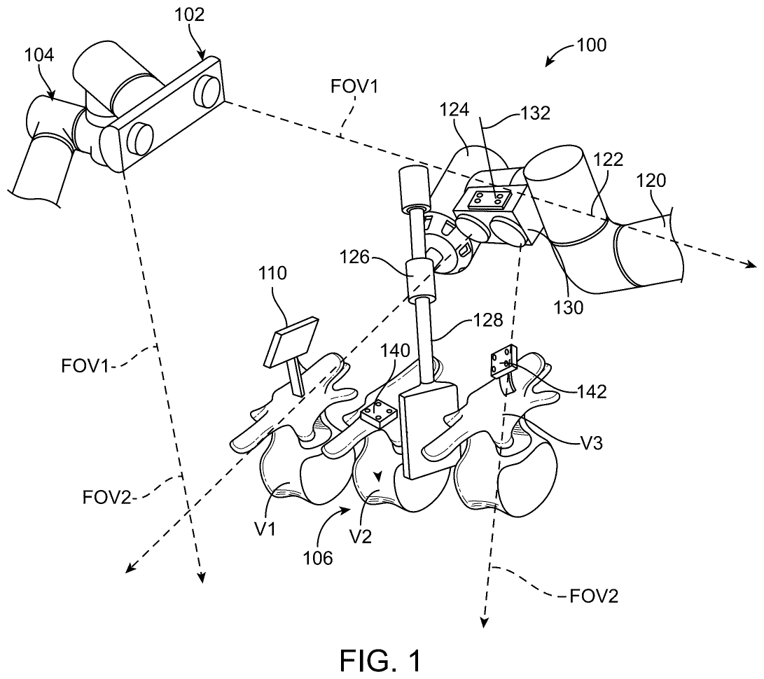

As shown in FIG. 1, is a representative surgical robotic system 100 that comprises a primary or surveillance navigation camera 102 mounted on a distal end of a surveillance robotic arm 104, in accordance with some embodiments. The base end of the surveillance arm 104 may be mounted on a robot platform (not shown) comprising, consisting of, or consisting essentially of a single cart or chassis as described in commonly owned PCT application PCT/IB2022/052297 (published as WO2022/195460), the full disclosure of which is incorporated herein by reference.

The surveillance arm 104 may be configured to hold the primary camera 102 at a conventional distance from a surgical site or field 106, typically in a range from 1.5 m to 2.5 m in a robotic spinal procedure as illustrated so that a field of view FOV1 of the primary navigation camera 102 may include most or all of the robotic components as well as most or all of the surgical site. In some embodiments, the field of view FOV1 of the primary navigation camera 102 are able to track a primary navigation marker 110 which is typically used for initial registration of the robotic coordinates to the patient anatomy and/or an initial pre-op patient CT or other scan. In some embodiments, the primary navigation marker will usually be relatively large, for example having an area greater than 10 cm2.

As further shown in FIG. 1, the robotic system 100 comprises at least one “working” robotic arm 120 having a distal end 122 which supports an end effector 124 comprising a gripper 126 which carries a surgical tool, such as a paddle 128. The gripper 126 can carry a variety of other surgical tools as well, such a cannula for implanting pedicle screws as described in commonly owned PCT publication WO2023/223215, the full disclosure of which is incorporated herein by reference.

One of skill in the art will understand, that while a single working surgical arm 120 is shown, two, three, fours, or even more working arms could be incorporated into the robotic systems of the disclosed technology.

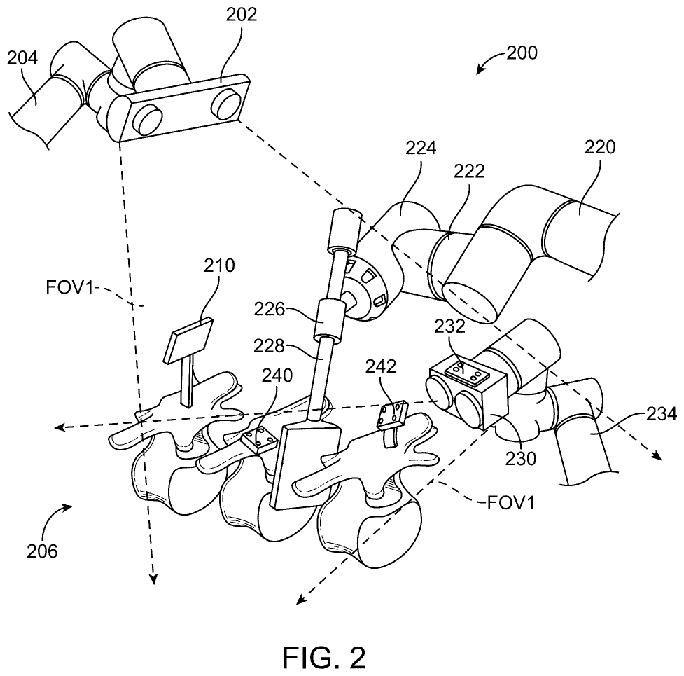

As described in the detailed description to this point, the robotic system is generally as described in PCT Publications WO2022/195460 and WO2023/223215, the full disclosure of which were previously incorporated herein by reference. The robotic systems of the disclosed technology differ, however, in that they are configured to deploy a secondary navigation camera which is intended to be positioned much closer to the surgical site 106, typically as close as 30 cm or less. The secondary navigation camera could be mounted on a “dedicated” robotic arm or on a working arm that also carries and deploys various surgical tools. In some instances, the secondary cameras will be “add-on” devices that can be detachably secured to the working or other robotic arms and/or the surgical tools or end effectors. A first example where a secondary navigation camera is mounted on a working surgical arm is shown in FIG. 1, and a second example where a secondary navigation camera is mounted on a dedicated secondary surveillance arm is shown in FIG. 2.

As shown in FIG. 1, a secondary navigation camera 130 may be removably or fixedly attached (usually removably) at or near the distal end of 122 of the working robotic arm 120. The secondary navigation camera may be smaller than the primary navigation camera and have a maximum dimension no greater than 10 cm and will be configured to focus on a narrower field of view FOV2 at a closer distance. In specific instances, the secondary navigation camera 130 will have one or more “camera” navigation markers 132 placed on it, and the markers 132 may be small, usually having a maximum dimension of 5 cm or less. Such small dimensions may allow the secondary navigation camera 130 with the attached navigation marker 132 to be manipulated by the working robotic arm 120 with minimal interference with the surgical procedure, e.g., reducing or eliminating interference with operation of the working arm 120 and/or with an operator's view of the surgical site 106.

FIG. 1 also shows the placement of additional “secondary” navigation markers used in performing a robotic surgical procedure. The primary navigation marker 110 may be secured to a first vertebra V1, prior to the procedure so that it can be used in a conventional registration of the patient anatomy. Secondary navigation markers 140 and 142 may be deployed on vertebrae V2 and V3, respectively. While these navigation markers could be implanted prior to a procedure and used in registration, more usually they will be deployed during the procedure, e.g., after the patient's spine has been progressively exposed, using conventional bone attachment modalities.

The secondary markers 140 and 142 (and additional secondary markers as needed) may be “miniaturized.” having a maximum dimension of 2 cm or less and be located at positions inside the patient's body, e.g., lying below the patent's skin surface adjacent to the surgical incision exposing the spine. As the markers 140 and 142 are effectively “buried” beneath the skin surface, the secondary navigation markers may be invisible to the primary navigation camera 102 in contrast to the larger primary navigation marker 110 which extends above the patient's skin.

Referring to FIG. 1, the camera navigation marker 132 deployed on the secondary navigation camera 130 may be visible to the primary surveillance camera 102, allowing the controller to optically track the position of the secondary navigation camera in and around the surgical site 106. While the position of the secondary navigation camera 130 could be kinematically tracked by the controller based on the kinematics of the working robotic arm 120, such kinematic tracking may be less preferred as such kinematic tracking is more difficult to implement to achieve a desired accuracy.

Information from the primary surveillance navigation camera 102 and the secondary navigation camera 132 can be used to optically register and subsequently track the positions of the primary navigation marker 110 and the first and second secondary navigation markers 140 and 142 (as well as any additional secondary navigation markers that might later be deployed). The primary navigation marker 110 may be optically tracked by the primary surveillance navigation camera 102 whose position is kinematically tracked the controller, as described in WO2022/195460 and WO2023/223215, the full disclosures of which were previously incorporated herein by reference. Simultaneously or substantially simultaneously, the primary surveillance navigation camera 102 can track the position of the secondary navigation camera 130 based on observing the camera navigation marker 132 which will be within the field of view FOV1 of the camera 102.

The position of secondary navigation markers 140 and 142, in turn, can be tracked by the secondary navigation catheter 130, and the controller can calculate the positions of markers 140 and 142 within the surgical coordinate spaced based on the position of the camera 130 within the surgical coordinate spaced. In this way, the controller can both register and track the positions of the secondary markers within the surgical coordinate space without the need to rely on kinematically calculated “pose” information, although use of pose information is not excluded from the disclosed technology.

FIG. 2 illustrates an alternative surgical robotic system 200 constructed in accordance with the principles of the disclosed technology, in accordance with some embodiments. The primary navigation camera 202 and primary surveillance arm 204 may be arranged to view a primary navigation marker 210 in a surgical site or field 206 generally as described for similar components in robotic system 100. The robotic system 200 differs mainly in that a secondary navigation camera 230 is mounted on a dedicated secondary surveillance arm 234 rather than on a working robotic arm 220 which carries only the end effector 224, gripper 226, and surgical tool 228 on the distal end 220 of the arm. Mounting of the secondary navigation camera 230 on the dedicated robotic arm 234 may be advantageous in that the secondary camera 230 can be positioned to view secondary navigation markers 240 and 242 at all times without regard to positioning of the surgical tool 228 mounted on working arm 220. The camera navigation marker 232 is shown to be mounted on the secondary navigation camera 230 but can be located elsewhere on the arm 234 so long as it the marker motion is representative of the camera motion.

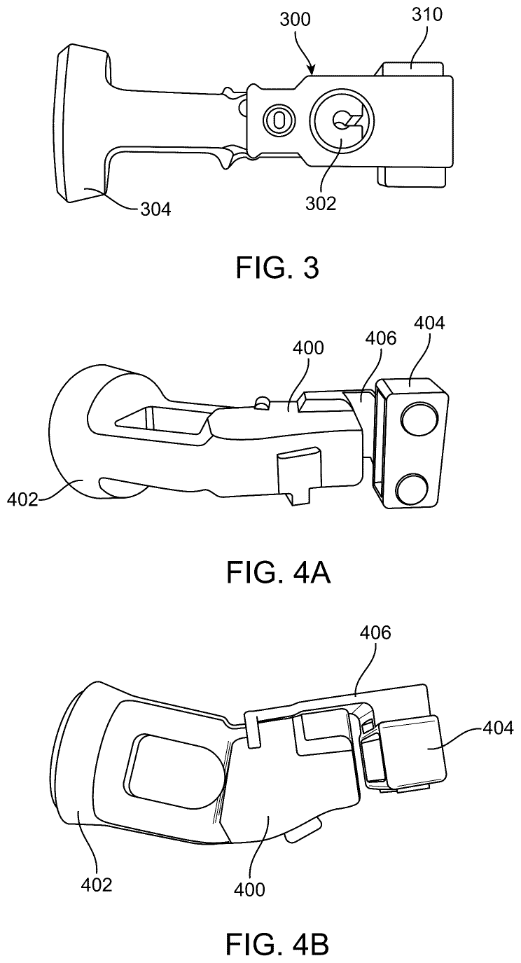

With reference to FIGS. 3, 4A, and 4B, the mounting of the secondary navigation cameras on tools and robotic end effectors is shown, in accordance with some embodiments. As shown in FIG. 3, an end effector 300 includes a gripper mechanism 302 and a mounting flange 304. A secondary navigation camera 310 may be mounted on a free end of the gripper tool 302 opposite to the mounting flange. The dimensions of a secondary navigation camera 310 may be minimized, usually having a maximum dimension no greater than 10 cm so as to reduce the chance of interfering with the surgical procedure, e.g., with operation of one of the robotic arms or with the surgical staff's view of the surgical field.

FIGS. 4A and 4B show an alternative secondary camera mounting embodiment including an end effector 400 and a mounting flange 402 configured to be attached to a free end of a robotic surgical arm (not shown). A secondary navigation camera 404 may be attached to the end effector 400 by a bracket 406. The bracket 406 may be configured to attach to a robotic arm in place of another tool, end effector, gripper or the like. In that way, the secondary camera(s) may be placed in different arrangements on a multi-armed surgical robot in accordance with the disclosed technology. In other instances, the bracket 406 may be configured to attached to a dedicated navigation or surveillance arm which will typically be free of other tools, end effectors, grippers and the like, as shown for example in FIG. 2.

While the secondary markers described thus far are usually smaller in size than the primary markers so the secondary markers will usually avoid or lessen interference with optical or other scanning of a surgical field, in some it will de desirable to still further reduce such interference. Such further interference reduction can be achieved by providing secondary markers having removeable trackable markers. The trackable markers are directly or indirectly attached to a bone anchor which remains in place during most or all of an entire robotic surgical operation where a trackable portion of the secondary marker can be detached and reattached at different times during the operation to minimize interference when desired while still allowing the secondary marking function to be restored when desired.

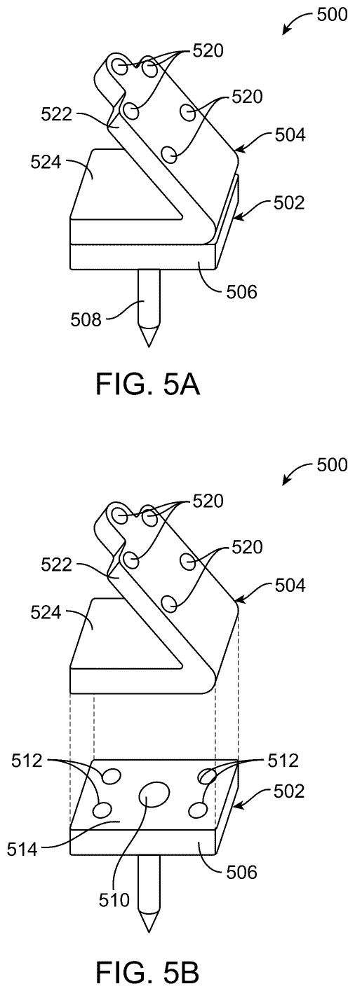

FIGS. 5A and 5B illustrate a surgical marker 500 in accordance with the principles of the disclosed technologies. The surgical marker 500 includes an implantable base 502 and a trackable marker 504 detachably secured to the implantable base. The implantable base 502 is shown with the trackable marker 504 attached in FIG. 5A and detached FIG. 5B. The base 502 includes an attachment interface 506, typically but not necessarily formed as a flat plate, having a bone anchor 508 on a lower surface thereof. The bone anchor is shown with a bone-penetrating tip so that the base 502 can be temporarily implanted at a bony target site at the beginning of an operation and removed from the target site after the operation is completed. Alternatively, the bone anchor 508 could comprise a clamp or other non-penetrating feature for temporary attachment to the bony anatomy. In all cases, the implantation/attachment of the base 502 to the bony anatomy will be very stable and resist displacement as an operation proceeds and the trackable marker 504 is detached and reattached.

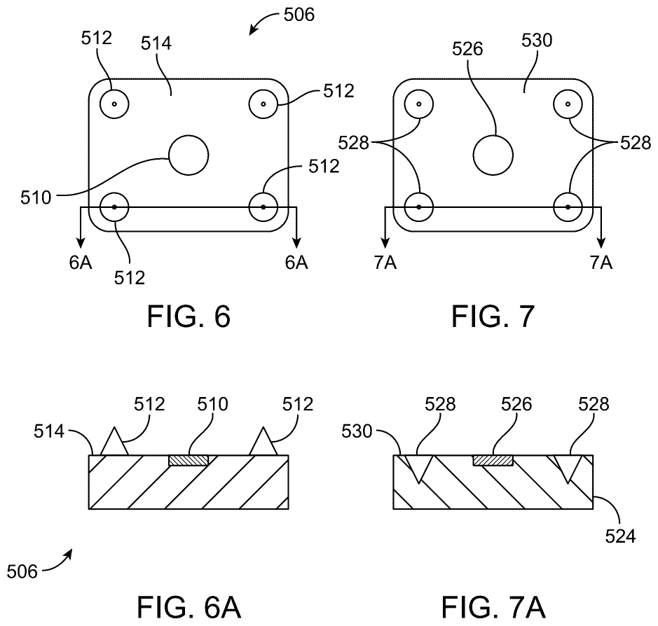

In attaching, detaching, and reattaching the trackable marker 504 from the base 502, it is necessary that that the attachment be stable and that their relative orientations be preserved. To achieve those objectives, both attachment and alignment features are provided, as shown in FIGS. 6, 6A, 7, and 7A. For example, an attachment magnet 510 and four alignment features 512 may be provided on an upper surface 514 of the attachment interface 506 of the base 502. A second magnet 526 and four alignment features 528 are provided on a lower surface 530 of the trackable marker 504. The magnets 510 and 526 and the alignment features 512 and 528 will be arranged in identical patterns so that they mate when the trackable marker 504 is mounted on the base 502 and hold the trackable marker and the based in a fixed, repeatable orientation. At least one of the magnets 510 and 526 will be a permanent magnet, for example a neodymium or other high strength magnet, while the second can be magnetizable or a magnet of the opposite polarity. The alignment feature will typically be mating elements, such a cone and a conical divot, as illustrated. Numerous other types of alignment features would also be useful.

Referring again to FIGS. 5A and 5B, the trackable marker 504 will typically be include an upper portion 522 and a lower portion, typically being flat plates joined at an angle selected to enhance visibility of a plurality of radiopaque or other detectable features 520 formed on the portion 522. While shown to be inclined relative at an axis of the bone anchor 508, in other instances the detectable features 520 could the arranged in a plane parallel or normal to the axis of the bone anchor.

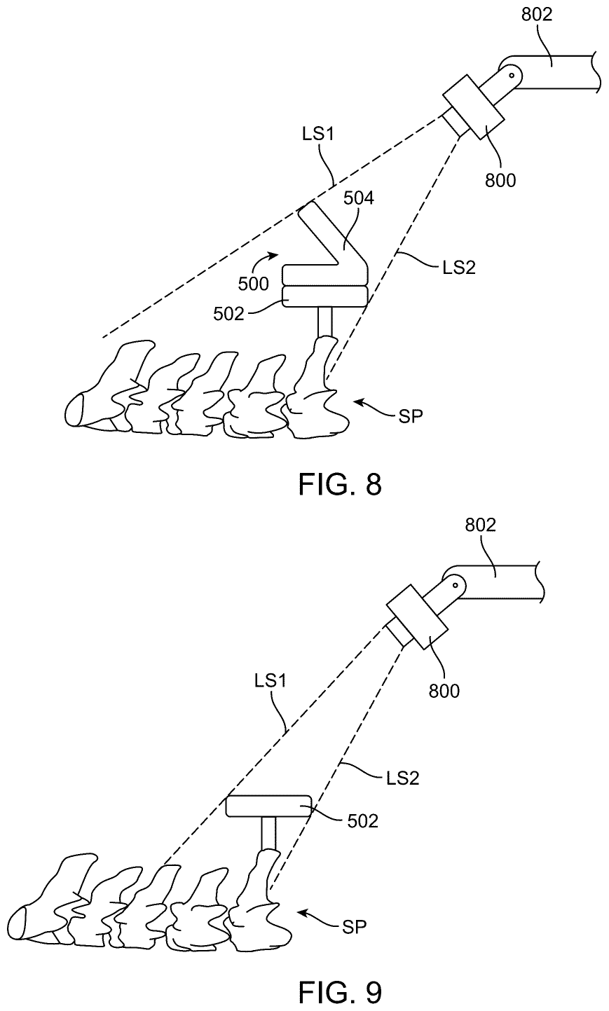

FIGS. 8 and 9 show the effect on “line-of sight” imaging provided by removal of the trackable marker from the base during a surgery. FIG. 8 shows that the “blind area” between sight lines LS1 and LS2 of camera 800 on surgical arm 802 is much larger in when the fully assembled surgical marker 500 is in place than in FIG. 9 after the trackable marker 504 has been removed, leaving only the base 502 in place. When needed, for navigation or other purposes, though, the trackable marker 504 can be easily reattached to the base 502.

Reference Numbers.

| | |
|---|---|
| 100 | Surgical robotic system |
| 102 | Navigation camera |
| 104 | Surveillance arm |
| 106 | Surgical field |
| 110 | Primary navigation marker |
| 120 | Working robotic arm |
| 122 | Distal end |
| 124 | End effector |
| 126 | Gripper |
| 128 | Surgical tool |
| 130 | Secondary navigation camera |
| 132 | Navigation markers |
| 140 | Secondary navigation marker |
| 142 | Secondary navigation marker |
| 200 | Surgical robotic system |
| 202 | Primary navigation camera |
| 204 | Primary surveillance arm |
| 206 | Surgical field |
| 210 | Primary navigation marker |
| 220 | Working robotic arm |
| 224 | End effector |
| 226 | Gripper |
| 228 | Surgical tool |
| 230 | Secondary navigation camera |
| 234 | Secondary surveillance arm |
| 240 | Secondary navigation marker |
| 242 | Secondary navigation marker |
| 300 | End effector |
| 302 | Gripper mechanism |
| 304 | Mounting flange |
| 310 | Secondary navigation camera |
| 400 | End effector |
| 402 | Mounting flange |
| 404 | Secondary navigation camera |
| 406 | Bracket |
| 500 | Surgical marker |
| 502 | Base |
| 504 | Trackable marker |
| 506 | Attachment interface |
| 508 | Bone anchor |
| 510 | Magnet |
| 512 | Alignment features |
| 514 | Upper surface |
| 520 | Detectable features |
| 522 | Upper portion |
| 524 | Lower portion |
| 526 | Magnet |
| 528 | Alignment features |
| 530 | Lower surface |
| 800 | Camera |
| 802 | Robotic arm |

In addition to the robotic systems just described, the disclosed technology also provides navigation methods for performing robotic surgical procedures. In some embodiments, the methods may employ a surgical robot having one robotic arm with a dedicated surveillance or navigation camera mounted thereon where a distal end of the surveillance arm may be positioned at a conventional distance from a surgical site on a patient, typically 1.5 m to 2.5 m. The surgical robot may additionally have a surgical arm configured with an end effector that can hold a surgical tool to perform a robotic procedure at the surgical site of the patient. In some embodiments, the surgical arm may also carry secondary navigation camera, typically a small secondary navigation camera having a maximum diameter 10 cm or less. Such a surgical robot is shown and described with reference to FIG. 1 herein.

The small, secondary navigation catheter may be attached to a distal end or region of the of the surgical arm, e.g., to the end effector, and can be positioned to have a direct view into the surgical site. The method may further comprise implanting a plurality of navigation markers, e.g., a relatively large navigation marker that may also reused for performing an initial registration step, as well as one, two, three, or more secondary navigation markers. For example, in spinal procedures, two, three, or more secondary markers may be used. Such multiple secondary cameras will usually be visible to the one or more secondary cameras even when invisible to the primary surveillance camera. Even when invisible to the primary navigation camera, the secondary navigation markers may be visible to the smaller, secondary navigation catheter which can be moved and positioned in the surgical site to view the secondary markers.

In some embodiments, in spinal procedures, the secondary markers may typically be placed on vertebrae of the patient so as to be visible to the secondary navigation camera, often being located within a surgical cavity that blocks viewing by the primary navigation camera. A primary navigation marker, typically larger than the secondary markers, may be placed on an adjacent vertebra and extend out of the surgical cavity so as to be visible to the primary surveillance navigation camera, and one navigation marker may be placed on the secondary navigation camera attached, for example, to the end effector on the surgical arm.

In this method, the primary surveillance navigation camera can see the navigation marker placed on the secondary navigation camera on the surgical arm and can also see the “anatomical” primary navigation marker extending out of the surgical site. The robotic controller mediates the movements of the surveillance and surgical arms and can do so with the navigation information provided, typically without the use of pose information provided by an initial registration step and subsequent kinematic tracking the robotic arms. Optical tracking can be continued when the primary surveillance navigation camera maintains a simultaneous view of the primary navigation marker and the marker on the secondary navigation camera. In some embodiments, the surgical robotic system has a dedicated secondary surveillance robotic arm with the secondary navigation camera mounted thereon in addition to the primary navigation camera, on the primary surveillance arm. A working surgical arm configured with an end effector may hold a surgical tool to operate at a surgical site of a patient. The secondary navigation camera on the dedicated secondary surveillance arm can be independently moved close to the surgical site of the patient in order to gain an advantageous view of the surgical tools and secondary markers without regard for the positioning and operation of the surgical tool.

In some embodiments, a registration step can be carried out at the beginning of the spinal surgical procedure. In some embodiments, the navigation marker that is mounted to a vertebra but is configured to extend out of the surgical site may incorporate radiopaque components that are visible to CT or x-ray.

In some embodiments, a registration step may be carried out so that the coordinate system of the robotic system is registered with the anatomy of the patient. If such a registration step is carried out, then the use of pose information for navigation is also possible during the surgical procedure. However, as discussed herein, the skilled artisan will understand that such pose information is not necessary. For example, a surveillance navigation camera may maintain a simultaneous view of a navigation marker attached to patient anatomy and extending from a surgical site and a navigation marker mounted to a miniature navigation camera mounted to an end effector, such that the miniature navigation camera is able to see anatomy elements adjacent to the patient anatomy on which is mounted the navigation marker extending from the patient anatomy that is visible to the surveillance navigation camera.

One of skill in the art will understand that variations on the described embodiments are possible, while still staying within the spirit of the disclosed technology. For example, the disclosed systems and methods could be deployed with surgical robotic systems with a surveillance arm and multiple surgical arms. In addition, the disclosed systems and methods could be deployed in surgical fields beyond spinal surgery. Any robotic surgical field that would benefit from coordinated navigation, no need for registration, and accurate navigation that does not interfere with surgeon workflow or line of sight would benefit from the application of the currently disclosed systems and methods. While embodiments of the disclosed technology have been shown and described herein, it will be obvious to those skilled in the art that such embodiments are provided by way of example only. Numerous variations, changes, and substitutions will now occur to those skilled in the art without departing from the scope of the invention. It should be understood that various alternatives to the embodiments described herein may be employed in practicing the invention. It is intended that the following claims define the scope of the technology and that methods and structures within the scope of these claims and their equivalents be covered thereby.

What is claimed is:

1. A method for performing a robotic surgical procedure, the method comprising:
providing a surgical robot with at least a first robotic arm, a second robotic arm, a first camera or other sensor on the first robotic arm, a second camera or other sensor on the second robotic arm, and a controller configured to receive images from the first and second cameras or other sensors and to kinematically position the first and second robotic arms;
placing a primary marker at a primary location on an anatomy of a patient;
scanning the patient anatomy and the primary marker with the first camera or other sensor to generate a primary image, wherein the controller registers the location of the primary marker within a coordinate system of the surgical robot based upon the primary image;
placing one or more secondary markers on secondary location(s) on the patient anatomy; and
scanning the one or more secondary markers with the second camera or other sensor to generate a secondary image, wherein the controller registers the secondary locations relative to the primary location within the coordinate system of the surgical robot based upon the secondary image.

2. The method of claim 1, wherein registering the secondary locations relative to the primary location comprises determining the location of the secondary camera or other sensor relative to the primary camera or other sensor.

3. The method of claim 2, wherein determining the location of the secondary camera or other sensor relative to the primary camera or other sensor comprises scanning the secondary camera or other sensor with the primary camera or other sensor.

4. The method of claim 2, wherein determining the location of the secondary camera or other sensor relative to the primary camera or other sensor comprises the controller kinematically determining locations of the first and second robotic arms.

5. The method of claim 1, further comprising continuing to scan the primary marker with the first camera or other sensor during subsequent portions of the robotic surgical procedure to track the primary location in the coordinate system of the surgical robot.

6. The method of claim 1, further comprising continuing to scan the one or more secondary markers with the second camera or other sensor during subsequent portions of the robotic surgical procedure to track the secondary location(s) in the coordinate system of the surgical robot.

7. The method of claim 1, wherein tracking the secondary location(s) in the coordinate system of the surgical robot comprises tracking a position of the second camera or other sensor relative to the first camera or other sensor.

8. The method of claim 1, wherein the primary marker is larger than the one or more secondary markers, and wherein the primary camera or other sensor when scanning the primary marker is at a distance from the primary marker which is greater than a distance of the secondary camera or other sensor from the one or more secondary markers when scanning the one or more secondary markers.

9. The method of claim 1, wherein the at least the first and second robotic arms are mounted on a common chassis which establishes the coordinate system.

10. The method of claim 1, wherein the primary marker is affixed to a primary vertebra and the one or more secondary markers are affixed to secondary vertebra.

11. The method of claim 1, wherein surgical tools are operated by one or more of the first or second robotics arms.

12. A method for performing a robotic surgical procedure, the method comprising:
providing a surgical robot with at least a first robotic arm, a second robotic arm, a first camera or other sensor on the first robotic arm, a second camera or other sensor on the second robotic arm, and a controller configured to receive images from the first and second cameras or other sensors and to kinematically position the first and second robotic arms in a surgical coordinate space;
kinematically tracking a location of the first camera or other sensor in the surgical coordinate space;
optically tracking a location of the second camera or other sensor in the surgical coordinate space with the first camera or other sensor; and
optically tracking one or more location(s) in the surgical coordinate space of one or more secondary markers affixed at one or more secondary location(s) on a patient anatomy with the second camera or other sensor;
wherein the controller calculates the one or more location(s) in the surgical coordinate space of the one or more secondary markers based the kinematically tracked position of the first camera or other sensor and the optically tracked one or more location(s) of the secondary markers relative to the second camera or other sensor.

13. A robotic surgical system comprising:
a surgical robot comprising at least a first robotic arm, a second robotic arm, a first camera or other sensor on the first robotic arm, and a second camera or other sensor on the second robotic arm; and
a controller configured to (a) receive images from the first and second cameras or other sensors, (b) kinematically position the first and second robotic arms in a surgical coordinate space, (c) kinematically track a position of the first camera or other sensor in the surgical coordinate space, (d) optically track a position of the second camera or other sensor using the first camera or other sensor, and (e) calculate positions of secondary markers in a field of view of the second camera or other sensor based on the position of the second camera or other sensor being tracked by the first camera or other sensor.
